(19) United States
(12) Patent Application Publication
(10) Pub. No.: US 2005/0038361 A1
(43) Pub. Date: Feb. 17, 2005

(54) Apparatus for improved shock-wave lithotripsy (SWL) using a piezoelectric annular array (PEAA) shock-wave generator in combination with a primary shock wave source

(75) Inventors: Pei Zhong, Chapel Hill, NC (US); Xufeng Xi, Sunnyvale, CA (US); Franklin H. Cocks, Durham, NC (US); Glenn M. Preminger, Chapel Hill, SC (US)

Correspondence Address: JENKINS & WILSON, PA, 3100 TOWER BLVD, SUITE 1400, DURHAM, NC, 27707, US

(73) Assignee: Duke University

(21) Appl. No.: 10/641,349

(22) Filed: Aug. 14, 2003

(51) Int. Cl. A61B 17/22

(52) U.S. Cl. 601/4

(57) ABSTRACT

The invention relates to an improved apparatus for the comminution of concretions in vivo by controlled, concentrated cavitation energy using two shock wave pulses with a specified time delay and pressure relationship, with the first shock wave pulse being used to induce a transient cavitation bubble cluster near the target concretion, and the second shock wave pulse to control and force the collapse of the cavitation bubble cluster towards the target concretion with concentrated energy disposition while avoiding injury to surrounding tissue. The invention contemplates the use of an improved combined electrohydraulic or electromagnetic and a piezoelectric annular array shock wave generator to produce improved stone comminution with reduced tissue injury in vivo.

### FIELD OF INVENTION

The present invention relates to a method for disintegration of concretions in vivo with reduced tissue injury, by the forced concentration of acoustically induced transient cavitation energy towards the target concretion through use of a piezoelectric annular array shock-wave generator of particular design in combination with a primary shock wave source.

### BACKGROUND OF THE INVENTION

Comminution of concretions in vivo using extracorporeally generated shock waves (lithotripsy) is a relatively recent medical practice, particularly in the treatment of urinary stone and biliary stone disease. Prior art describes various devices and methods for generating high-intensity, focused shock waves for the fragmentation of concretions inside a human being. U.S. Pat. No. 3,942,531 by Hoff et al. discloses the use of a spark gap discharge in water to generate a shock wave within an ellipsoidal reflector which couples and focuses the shock wave to fragment kidney stones inside the body. Hahn et al. in U.S. Pat. No. 4,655,220 disclose a device using a coil and a mating radiator, in the form of spherical segment, to produce magnetically induced self-converging shock waves. Wurster et al. in U.S. Pat. Nos. 4,821,730 and 4,888,746, disclose the use of piezoelectric elements arranged in mosaic form on a spheroidal cap to produce focused high-intensity shock waves at the geometric center of the cap, where the concretion must be placed. Many other shock wave generating systems are known in the art.

Despite the different principles used for shock wave generation, all of these devices produce shock waves of a similar waveform, which can be characterized by a compressive phase consisting of a rapid shock front with a positive peak pressure up to 100 MPa, followed by a rarefaction (negative) phase with a negative peak pressure up to 10 MPa and with a few microseconds duration. It is also well known in the lithotripsy art that the negative phase of an incident shock wave can induce transient cavitation bubbles in the focal region.

It is further known in the lithotripsy art that when cavitation bubbles collapse near a stone surface, microjets will be produced due to the asymmetric collapse of the cavitation bubbles. These microjets impinge violently onto the stone surface and cause stone fragmentation. Experiments have shown that using the same shock wave generator at the same intensity level, a stone immersed in glycerol (a cavitation inhibitive medium) will not be damaged, while the same stone immersed in an aqueous solution such as water (a cavitation promotive medium) can be fragmented, despite the fact that the transmission of the shock wave energy in both cases is the same. It is established in the lithotripsy art that shock wave induced cavitation and the resultant microjet impingement is one of the primary mechanisms for stone fragmentation. Furthermore, when shock wave-induced cavitation bubbles collapse near tissue surfaces, they can cause tissue injury through shock wave emission, the generation of high-temperatures, microjets, and the shear stresses associated with rapid bubble oscillation.

It has further been discovered in the past that the collapse of a cavitation bubble cluster can be controlled so as to cause increased concretion comminution by imposing an impinging shock wave of appropriate shape and intensity to collapse the bubble cluster from its outer layer into an inner layer collectively.

The collapse of a cavitation bubble by an impinging shock wave is found to be asymmetric, leading to the formation of a liquid jet which travels along the direction of the impinging shock wave. When occurring in water the liquid jet will be a water jet. It has been discovered in the past that the collapse of a cavitation bubble can be controlled and guided by an incident shock wave, provided that this shock wave is applied at the correct time in the life of a cavitation bubble. It is known in the art that the collapse of a cavitation bubble cluster by an impinging shock wave can concentrate 80% to 90% of the cavitation bubble energy from an outer layer to an inner layer, when these cavitation bubbles are forced to collapse in sequence by the incident shock wave. This concerted, controlled collapse of a cavitation bubble cluster by an impinging shock wave is found to produce an efficient concentration of the cavitation energy towards the center of the bubble cluster, where the concretion is located. Because the cavitation energy is directed towards and concentrated on the target concretion, tissue injury associated with the comminution of the concretion is reduced. Therefore, the comminution of concretions in vivo utilizing controlled, concentrated cavitation energy has the advantage of increased fragmentation efficiency with reduced tissue injury.

Similarly, Cathignol et al. in U.S. Pat. No. 5,219,401 disclose an apparatus for the selective destruction of biological materials, including cells, soft tissues, and bones. The injection of gas bubble precursor microcapsules, having diameters preferably in the 0.5 to 300 micron range and made from materials such as lecithin, into the blood stream is used by Cathignol et al. as the primary means of generating gas bubbles in vivo. Although the phenomenon of cavitation provoked by an ultrasonic wave generator working in a frequency range of 10 to 100 kHz is described, the sonic pulse sequence is not specified. As it has been discovered in the lithotripsy art, the forced collapse of cavitation bubbles to produce fluid microjets for the enhanced comminution of concretions requires a specified relationship between the first, cavitation-inducing, acoustic pulse and the second, cavitation-collapsing, acoustic pulse. In addition, it has been discovered that the second, cavitation-collapsing, acoustic pulse must have a compressive (positive) phase with a long duration and only a small, or no, tensile (negative) component.

Reichenberger, in U.S. Pat. No. 4,664,111, discloses a shock wave tube for generating time-staggered shock waves by means of a splitting device, such as a cone, for the fragmentation of concrements in vivo. Reichenberger discloses that the effects of the shock waves can be improved if they are so closely spaced in time that they overlap in their action on the concrement. The effects of shock wave induced cavitation are not considered or mentioned by Reichenberger.

Thus, none of the prior art described hereinabove teaches the use of a secondary shock wave, imposed at a specified time delay, to control the collapse of a transient cavitation bubble cluster induced by a primary shock wave. Without this time sequenced second shock wave, it has been discovered that the efficiency of comminuting concretions in vivo by shock wave lithotripsy will be low, and the concomitant risk for tissue injury due to the uncontrolled cavitation energy deposition during the procedure will be correspondingly increased. However, there have been preliminary discoveries to date relating to this aspect of lithotripsy technology.

Of particular relevance to time sequenced secondary shock waves, Zhong et al. in U.S. Pat. No. 5,582,578 provides such a method for generating a sequence of shock wave pulses with a specified very short time delay (less than 400 microseconds), and with pressure relationships between the individual pulses that provide both a means of inducing a transient cavitation cluster, and a means of controlling the growth and subsequent collapse of the cavitation bubble cluster near the target concretions in vivo, to achieve increased fragmentation efficiency with reduced tissue injury.

Further relating to Zhong et al. U.S. Pat. No. 5,582,578, applicants have previously developed a shock wave generator comprising a piezoelectric annular array (PEAA) shock-wave generator that can be retrofitted on a clinical (for example, a DORNIER HM-3) lithotripter to generate a sequence of shock wave pulses. The PEAA generator was intended to produce an auxiliary shock wave to control and force the collapse of lithotripter-induced bubbles toward the target concretion for improved stone comminution. A prototype PEAA generator was combined with an experimental electrohydraulic (EH) shock-wave lithotripter with a truncated HM-3 reflector in previous experiments. Stone fragmentation tests in vitro were carried out and these results demonstrated that 60% to 80% increment in stone fragmentation could be achieved using the combined shock-wave generator with optimal interpulse delay.

The previous combined EH/PEAA shock wave generator was described in a paper entitled “Improvement of Stone Fragmentation During Shock Wave Lithotripsy Using a Combined EH/PEAA Shock Wave Generator - In Vitro Experiments” by Xi and Zhong, which was published in Ultrasound in Medicine and Biology. Volume Number 26, pages 457-467 in 2000 and showed that the collapse of cavitation bubbles induced during shock wave lithotripsy could be modified by the use of a secondary pulse produced by piezoelectric transducers made of piezoceramic (PZT-4) disks. Xi and Zhong found that in in vitro conditions stone comminution could be increased significantly when the secondary pulse produced by piezoelectric transducers occurred during the collapse phase of the cavitation bubbles produced by the primary shock wave generated by an electrohydraulic shock wave lithotripter. Xi and Zhong did not investigate the effects of their apparatus under in vivo conditions. Surprisingly, the method disclosed by Xi and Zhong has not been found to work in in vivo testing. While it is not known with certainty why the method of Xi and Zhong failed in in vivo testing it may be because the disruption during the passage of the auxiliary shock wave pulses produced by the piezoelectric transducers through tissue is too much greater than that which occurs under in vitro conditions. Clinical application inevitably requires the passage of the secondary shock wave pulses produced by the piezoelectric transducers through tissue. Furthermore, clinical application also requires the use of acoustic monitors and x-ray enhancing air sacks, which decrease the area available for piezoelectric transducers. In the apparatus described by Xi and Zhong all available space was used for piezoelectric transducers and Xi and Zhong do not disclose any means for allowing the effective clinical use of secondary shock wave pulses produced by the piezoelectric transducers to enhance stone comminution.

Thus, the previous known combination of a PEAA generator and an EH lithotripter suffers from certain shortcomings in the efficacy of its performance that have now become apparent to those skilled in the art. Applicants' discovery is believed to overcome these shortcomings and to provide an improved combined PEAA generator and EH generator.

### SUMMARY AND OBJECTIVES OF THE INVENTION

The present invention provides an improved apparatus and method for generating a sequence of shock-wave pulses with a specified very short time delay, and with pressure relationships between the individual pulses that provide a means of inducing a transient cavitation cluster, and a means of controlling the growth and subsequent collapse of the cavitation bubble cluster near the target concretions in vivo, to achieve increased fragmentation efficiency with reduced tissue injury. After extensive experimentation, it has now been discovered that a particular combination of electrohydraulic (EH) or electromagnetic (EM) primary shock wave generators and a piezoelectric annular array (PEAA) to generate a secondary shock wave pulse with a particular timing and arrangement with respect to the primary shock wave pulse will produce improved stone comminution in vivo with reduced tissue injury.

It is therefore an object of the present invention to provide an improved apparatus for producing controlled, concentrated collapse of cavitation bubbles for effective comminution of concretions in vivo with reduced injury to surrounding tissue by means of the combination of a primary shock wave pulse and a secondary shock wave pulse.

Some of the objects of the invention having been stated, other objects will become apparent from the following description of the drawings and appended claims.

### DESCRIPTION OF THE DRAWINGS

FIG. 1 (Prior Art) shows a concretion in a living body and a prior art shock wave generation system generating two shock wave pulses in sequence separated by a specified time delay for the comminution of concretions inside a living body;

FIG. 2 (Prior Art) shows two shock wave pulses in sequence separated by specified time delay of 50-400 microseconds (ps) to induce, by the tensile phase of the first shock wave pulse, a transient acoustic cavitation bubble cluster near a target concretion and to collapse, by the second shock wave pulse, the induced cavitation bubble cluster after it expands to its maximum size, to concentrate the cavitation energy in the form of liquid microjets towards the target concretion for improved fragmentation efficiency with reduced tissue injury (prior art);

FIG. 3 (Prior Art) is a front elevation view of a prior art combined electrohydraulic and piezoelectric annular array shock wave generator wherein the piezoelectric annular array generator consists of eight individual transducers arranged in an annular format with a supporting frame around the electrohydraulic (EH) generator and which uses a truncated DORNIER HM-3 reflector;

FIG. 4 (Prior Art) is a schematic diagram of an experimental lithotripter and an optical setup for shadowgraph and photoelastic imaging using the combined electrohydraulic and piezoelectric annular array shock wave generator shown in FIG. 3;

FIG. 5 (Prior Art) shows a graph of different acoustic emission signals produced by (a) the electrohydraulic generator shown in FIG. 3 at 24 kV and (b) the piezoelectric annular array generator shown in FIG. 3 at 15 kV; and

FIG. 6A is a schematic vertical cross-sectional view of the improved combined electrohydraulic (EH) and piezoelectric annular array (PEAA) generator of the present invention; and

FIG. 6B is a schematic front elevation view of the improved apparatus shown in FIG. 6A.

### DETAILED DESCRIPTION OF THE PREFERRED EMBODIMENTS

### A. Prior Art Combined EH and PEAA Generator

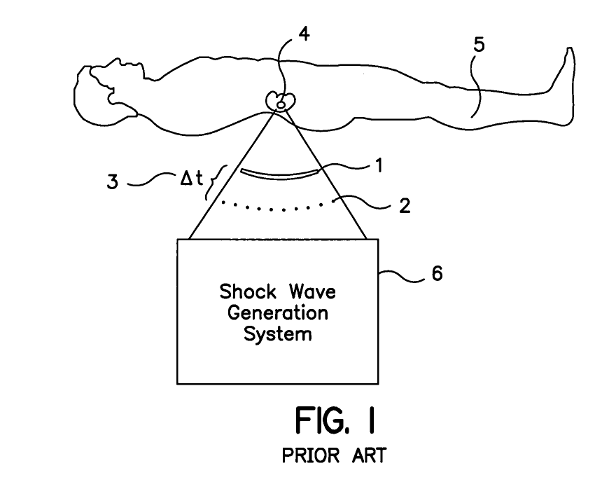

FIG. 1 shows a method of using two shock wave pulses 1, 2 separated by a specified time delay Δt 3. The shock wave pulses 1, 2 are produced by a shock wave generation system 6 and aimed confocally at a target concretion 4 inside a living being 5, for the comminution of the target concretion 4 with improved fragmentation efficiency and reduced tissue injury. These two pulses consist, respectively, of a first shock wave pulse 1 and second shock wave pulse 2, separated in time by a time delay Δt 3. It has been discovered that for optimal effect, this delay should be 50 to 400 microseconds (μs).

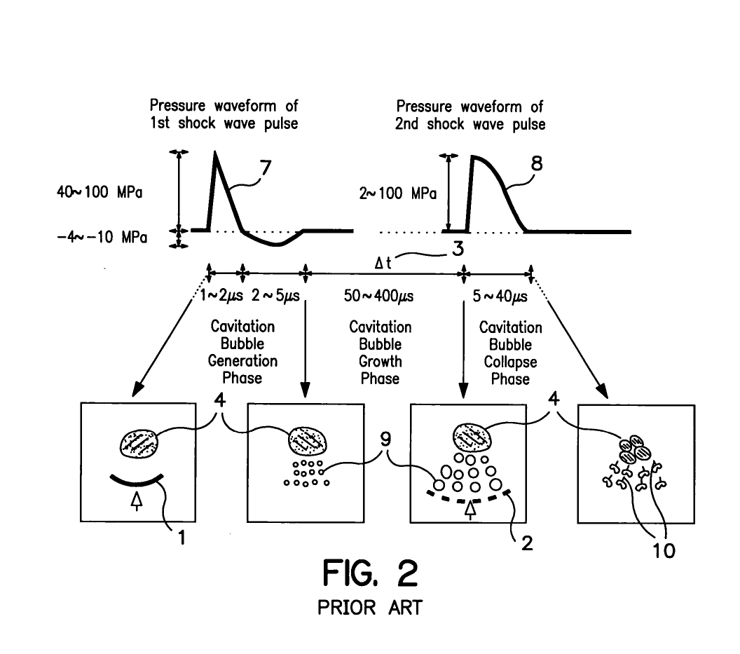

Also, another prior art technique is illustrated in FIG. 2, where the pressure waveform 7 of the first shock wave pulse 1 consists of a compressive phase with a positive peak pressure amplitude in the 20 to 100 million pascals (MPa) range and with a positive duration of 1 to 2 microseconds, followed by a tensile phase with a negative peak pressure amplitude of minus 1 to minus 10 MPa and with a duration of 2 to 5 microseconds. The pressure waveform 8 of the second shock wave pulse 2 consists of essentially a compressive phase with a positive peak pressure amplitude of 2 to 100 MPa and a duration of 5 to 40 microseconds. It was discovered that the time delay Δt 3 between the first shock wave pulse 1 and the second shock wave pulse 2 should be in a range of 50 to 400 microseconds for achieving improved stone comminution and reduction in tissue damage.

According to another advantageous embodiment of the prior discovery as shown in FIGS. 1 and 2, the tensile phase of the first shock wave pulse 1 is used to induce a transient cavitation bubble cluster 9 near a concretion 4 surface, with the induced cavitation bubble cluster 9 growing to its maximum size in 50 to 400 microseconds, depending on the intensity of the first shock wave pulse 1. The second shock wave pulse 2, separated from the first shock wave pulse 1 by a specified time delay is used to collapse the cavitation bubble cluster 9 at its maximum expansion, leading to a concerted collapse of the cavitation bubble cluster 9 towards the target concretion 4. This forced collapse has been found to result in the formation of high-speed liquid jets 10 impinging towards the target concretion 4 and to cause disintegration of the stone 4 with increased rapidity as compared to the uncontrolled collapse of the cavitation bubble cluster.

According to another embodiment of the prior discovery, the first shock wave pulse 1 can be generated by an electrohydraulic device, utilizing a spark gap discharge in water within an ellipsoidal reflector, such as the apparatus disclosed by Hoff et al. in U.S. Pat. No. 3,942,531. Electromagnetic shock wave generators, well known to those skilled in the art, may also be used such as the apparatus disclosed by Hahn et al. in U.S. Pat. No. 4,655,220. In addition, piezoelectric shock wave generators are equally well known to those skilled in the art and may also be used, such as the apparatus disclosed by Wurster et al. in U.S. Pat. No. 4,821,730. These previously disclosed devices generate a distribution of high-intensity shock waves in a focal volume embracing the target concretions 4. It is well known in the art that the beam diameter of the shock wave pulses in the focal plane and the depth of focus along the shock wave axis are in the range of 2 to 15, and 12 to 120 mm, respectively. It has also been discovered that the transient cavitation bubble cluster, induced by these devices, is distributed in a volume between 1.4 and 65 cubic centimeters.

According to another advantageous embodiment of the prior discovery, the second shock wave pulse 2 can be generated piezoeletrically by the superposition of individual shock wave pulses of different amplitudes, frequencies and phases, as disclosed by Wurster et al. in U.S. Pat. No. 4,888,746. Wurster et al. disclose a focussing ultrasound transducer comprising of mosaic assemblies of piezoelectric materials mounted on an inner surface of a spherical cap, with the energizing of individual piezoelectric elements being controlled electronically. Moreover, Wurster et al. disclose that by energizing in a particular sequence an array of piezoelectric elements, in such a manner that the negative halfwaves of the sound waves generated at the active transducer surface by momentary reverse oscillation of the transducer areas energized in each case may be balanced by an energizing in phase opposition of other transducer elements, meaning that a positive pressure surge only will be generated at the focal point.

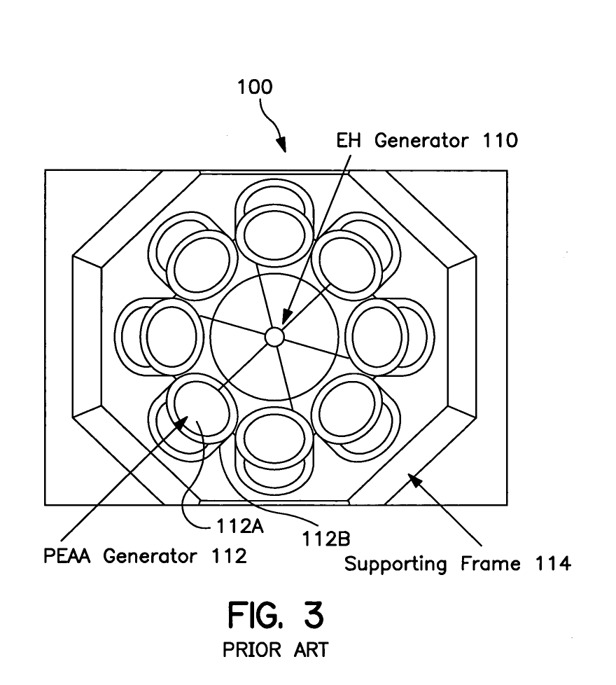

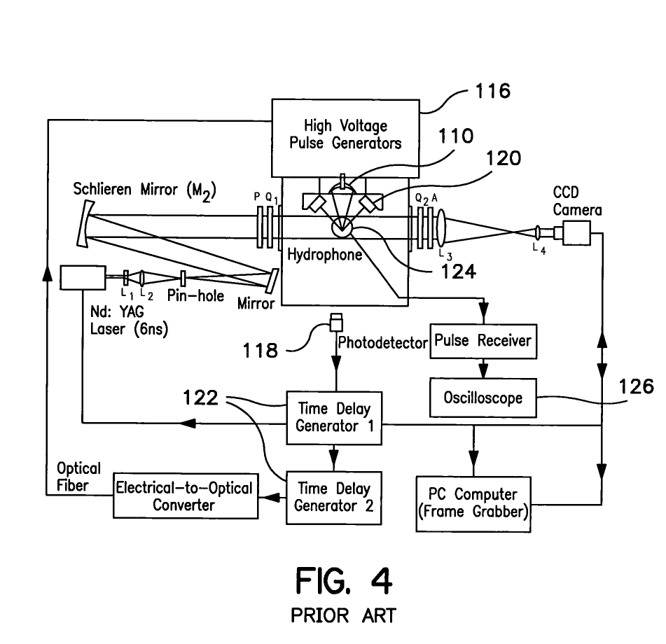

To assess cavitation control in a clinically relevant configuration, an experimental lithotripter utilizing a combined EH/PEAA shock-wave generator 100 (FIG 3) was previously designed and fabricated by applicants a Duke University in Durham, N.C. While the EH generator 110 was used to simulate the shock wave and associated cavitation produced by a clinical lithotripter, the added PEAA generator 112 was used to control the collapse of cavitation bubbles induced by the EH source. The prototype PEAA generator consisted of eight individual transducers 112 assembled in an annular format on a supporting frame 114 that connects mechanically to the EH source 110. Each transducer 112 was made of a disk-shaped PZT-4 element 112A (Channel Industries, Santa Barbara, Calif., D=50 mm, Thk=10 MM) and an aluminum disk (not shown) of the same size as backing material, with both fixed inside a Lucite cylinder 112B using epoxy resin (not shown). The PEAA generator 112 (focal length F=150 mm) was aligned coaxially and confocally with the EH source 110 that uses a truncated DORNIER HM-3 reflector (not shown) [semimajor axis a=138 mm, semiminor axis b=77.8 mm, and focal length (from aperture to F2)=190 mm], so that the total incident angle of the combined shock-wave generator 100 was about 105°, so as to be kept within the range used by clinical lithotripters. The combined shock-wave generator 100 was mounted horizontally in a Plexiglas tank (51×64×76, H×W×L cm) filled with degassed (O₂ concentration<4 mg/L) and deionized water. FIG. 4 shows a schematic diagram of the previously developed experimental lithotripter and the high-speed imaging system used for characterization of the in situ shock wave-bubble interaction generated by the combined EH/PEAA shock-wave generator 100.

The PEAA generators 120 and EH generator 110 were energized individually by two independent high-voltage pulse generators 116 of local design. The pulse generator for the PEAA source used a 0.5 μF capacitor and a discharge voltage adjustable between 10 and 20 kV; the pulse generator for the EH source used two 40-nF capacitors in parallel, and operated between 20 and 30 kV with a standard DORNIER electrode. In all the experiments reported, the PEAA generator 120 was operated at 15 kV, and the EH generator 110 at 24 kV, either individually or combined. Both generators were shielded and grounded to reduce the emission of electromagnetic noise produced by the high-voltage discharge. Moreover, trigger signals for the generators were provided by optical-to-electrical converters through optical fibers to prevent cross-talking between the two shock-wave sources in operation. In a typical cavitation control experiment, the EH source 110 was fired first. The spark discharge from the electrode was then picked up by a fast photodetector 118 (PDA450, Thorlabs, Newton, N.J.) and relayed through a digital delay generator 122 (DG535, Stanford Research Systems, Sunnyvale, Calif.) to provide a time-delayed signal to trigger the PEAA generator 120. The jitter for the PEAA generator 120 (time delay between the input trigger signal and output shock wave) was found to be less than 5 μs. Because bubbles induced by an EH lithotripter usually expand and then collapse within 200 to 400 μs, the shock wave produced by the PEAA generator 120 could be used reliably to interact with the bubbles at different stages of their oscillation.

The pressure waveform produced by either the PEAA 120 or EH 110 source individually was measured using a calibrated polyvinylidene difluoride (PVDF) membrane hydrophone 124 (Sonic Industries, Halboro, Pa.) that had a frequency bandwidth of 20 MHz, a minimal rise time resolution of 11 ns and a sensitivity of 6.8 kPa/mV. To map the acoustic field of the PEAA generator 120, the PVDF hydrophone was scanned at 1- or 2-mm steps, either along or transverse to the shock-wave axis. For the EH source 110, measurements were only carried out at the focal point. The output signal of the hydrophone was recorded on a LECROY digital oscilloscope 126 (Model 9314) at 100 MHz sampling rate.

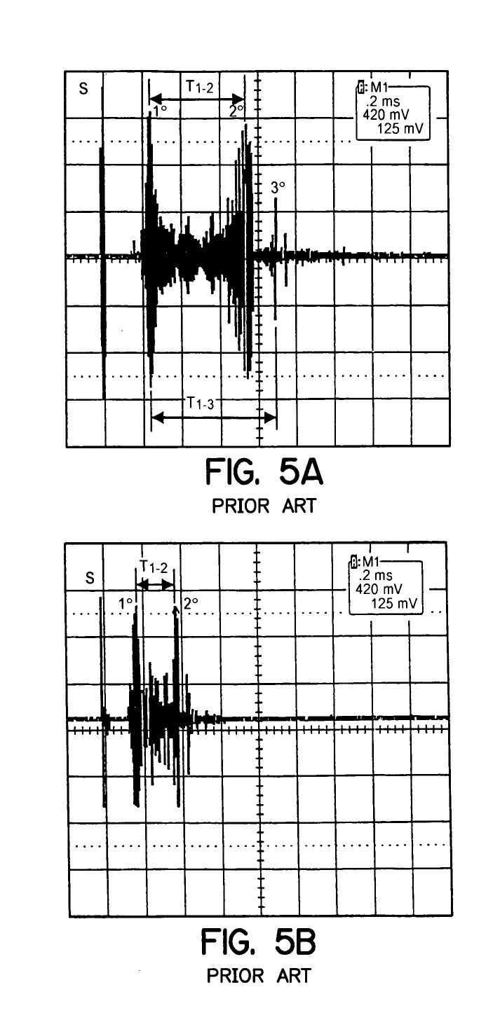

The duration of bubble oscillation induced by the EH 110 or PEAA generator 120 was determined using a passive cavitation detection system and a 2.25-MHz, resonant frequency focused hydrophone 124 (F=101.6 mm) was used. The -6-dB beam diameter of the focused hydrophone was estimated to be about 3 mm, so that bubble activity within a small volume around F2 could be detected. The focused hydrophone was aligned perpendicular to the lithotripter axis and confocally with F₂. FIG. 5 shows an example of the typical acoustic emission (AE) signals associated with the bubble oscillation produced by the EH 110 and PEAA 120 source, respectively. The first burst (1°) represents the initial compression and subsequent rapid expansion of pre-existing cavitation nuclei by the incident shock wave, whereas the second burst (2°) corresponds to the primary collapse of the bubble cluster. For the EH source 110, a distinctive third burst (3°), corresponding to the subsequent collapse of large rebound bubbles, could also be identified. Because of the distinct burst structure, the collapse time of the bubbles with respect to the arrival of the lithotripter shock wave at F2 (T₁₋₂ for the bubble cluster and T₁₋₃ for the rebound bubbles) could be easily measured. Subsequently, corresponding values for the EH source 110 were used to control the trigger of the PEAA generator 120, so that forced collapse of the bubbles could be produced at various stages of their oscillation.

Using a PEAA generator 120 that is combined with an experimental EH lithotripter 110, it was previously demonstrated in vitro that stone fragmentation could be significantly improved when appropriate shock-wave sequence was used. The auxiliary shock wave produced by the PEAA generator 120 was on the order of 8 MPa in peak positive pressure, which, acting by itself, is not sufficiently strong to produce stone fragmentation. However, when combined appropriately in time sequence with the EH lithotripter pulse, this auxiliary shock wave was found to greatly intensify the collapse of lithotripter-induced bubbles near the stone surface, leading to significantly improved stone comminution. The maximum increment in stone fragmentation could be achieved consistently for stone phantoms of three different densities, when the auxiliary shock wave was delivered to interact directly with the aggregated bubbles on the surface of the stone. However, it was surprisingly found that when used in experiments involving artificial kidney stones implanted into swine kidneys that the beneficial results previously observed in vitro did not occur in vivo.

### B. The Improved Electrohydraulic and Piezoelectric Annular Array

### Generator

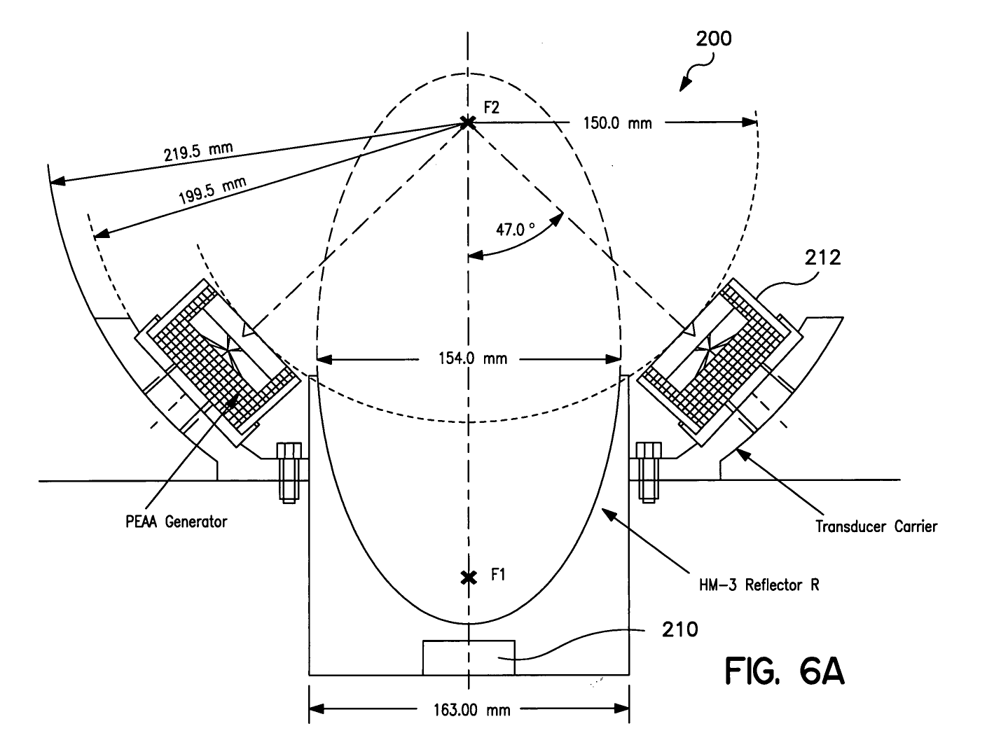

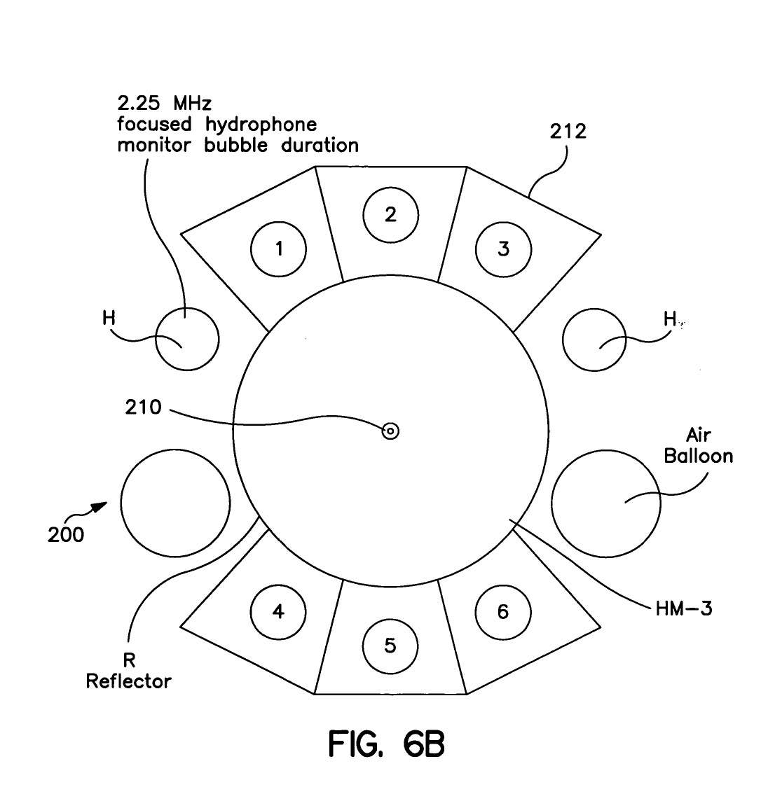

In a preferred embodiment 200 of the present invention as shown in FIGS. 6A and 6B, an array of six focused sets of piezoelectric elements 212 is positioned around the reflector R and the axis of a primary shock wave source 210 to form combined shock-wave generator 200 although between 6 and 2000 piezoelectric elements 212 could be used. Alternative positioning of the piezoelectric elements is also possible provided they are operatively associated with the circumference of the reflector of the primary shock wave source. In this preferred embodiment, the piezoelectric element consists of piezoceramics embedded in epoxy resin to form composite piezoelectric blocks. It has now been found that each individual composite piezoelectric block must be itself made spherically concave and focused on a convergence spot that is essentially congruent with the target concretion. Furthermore the ensemble of piezoelectric block elements must also be focused in such a way that each individually focused piezoelectric block element does not interfere with the output of any other piezoelectric block element. It has been discovered that the piezoelectric elements 212 are preferably arranged in a spherically concave configuration around the reflector R of the primary shock wave source 210. In this preferred embodiment, six such elements 212 are used. However, as few as two elements or as many as twenty elements 212 may be used. Spaces are also provided for the passage of x-rays for the localization of the kidney stones to be comminuted.

In the preferred embodiment peak pressure from 9 to 30 MPa is produced by the ensemble of piezoelectric elements 212 at the focus of the primary shock wave source 210. Importantly also, it is important that this peak pressure produced by the piezoelectric elements be produced within at least 401 μs, but less than 1000 μs after the peak pressure of the primary shock wave source is produced although a range of 10 μs to 1000 μs is possible.

In the preferred embodiment, the primary shock wave source 210 is an electrohydraulic spark generator. However, applicants contemplate that an electromagnetic shock wave generator can also be used. It is important that the primary shock wave source 210 produce a peak pressure of at least 20 MPa, but less than 130 MPa. Importantly also, the duration of the tensile component of the primary shock wave must be at least 2 μs, but less than 10 μs. The duration of the compressive component of the primary shock wave must be at least 0.5 μs, but less than 3 μs.

In the preferred embodiment, the array of piezoelectric elements 212 and the primary shock wave source 210 are additionally provided with at least two self-focused hydrophones H that are confocally aligned with the primary shock wave focus and with the piezoelectric shock wave focus. In this preferred embodiment, the self-focused hydrophones H are PANAMETRICS hydrophones whose focal length is 150 mm and whose nominal element diameters is 37.5 mm.

In operation, the preferred embodiment operates as follows: the primary shock wave source 210 is trigged to generate a shock wave that induces cavitation bubbles around the targeted kidney stones, which are located at the focus of the primary shock wave source. The duration of the bubble oscillation (expansion and collapse) is determined from the acoustic emission signals picked up by the two self-focused hydrophones H, which are aligned confocally with the primary shock wave source 210. This acoustic emission information is used to determine the interpulse delay between the shock waves generated by the primary shock wave source 210 and those generated by the piezoelectric elements 212. Improved stone comminution is achieved when the shock wave produced by the piezoelectric elements 212 arrive at the focus of the primary shock wave during the collapse phase of the cavitation bubbles produced by the primary shock wave 210. In this way, it has been found that intensified collapse of cavitation bubbles towards the target kidney stones is produced, leading to improved comminution of the targeted kidney stones.

In summary, prior research using a combined EH/PEAA shock-wave generator 100 with optimal pulse sequence resulted in significant enhancement in stone comminution in vitro. The results pointed to the possibility of utilizing such a concept for improving lithotripsy efficiency. Applicants have now discovered an improved apparatus of use utilizing a combined EH/PEAA shock-wave generator 200 comprising an improved PEAA array and configuration that results in unexpected and surprising enhancement in efficacy of the combined EH/PEAA shock-wave generator in vivo.

### C. Physics of the Improved Electrohydraulic and Piezoelectric Annular

### Array Generator

Shock wave lithotripters make use of the fact that the acoustic properties of human tissue are similar to those of water whereas the acoustic properties of renal concretions are very different from either water or tissue. Because of this, acoustic signals can be transmitted through water and tissue but be partially absorbed and partially reflected by a concretion. By focusing high-pressure acoustic impulses on a human concretion in a living body, the concretion may be fragmented by means of both sound pressure effects and cavitation bubble effects. It has been found that a secondary acoustic pulse, of intensity not high enough to cause stone fragmentation by itself, if properly timed with respect to the initial acoustic pulse, can cause the cavitation bubbles produced by the high-intensity initial pulse to collapse towards the concretion before reaching a size large enough to burst capillary vessels. It has now been discovered that an improved shock wave lithotripter apparatus for comminuting renal concretions may be made by combining a primary shock wave source, whether it is electrohydraulic or electromagnetic, with secondary shock wave sources.

In the electrohydraulic case, it has been discovered that the second shock wave sources of a particular type and arrangement, when mounted on the circumference of the reflector which is used to focus the acoustic impulses from the electrohydraulic shock wave source on renal concretions can under particular conditions produce improved stone comminution in vivo with reduced tissue injury. The primary shock wave source has a maximum pressure that produces cavitation bubbles around the focus of the primary shock wave source. By incorporating a plurality of piezoelectric generators of a particular type and arrangement, auxiliary shock waves can be produced of the right intensity and timing to cause beneficial effects on stone comminution while reducing kidney damage. These piezoelectric generators are oriented to have a common convergence spot, which is congruent with the focus of the primary shock wave source. Each of these piezoelectric generators consists of at least one spherically concave piezoelectric element. By making each piezoelectric element spherically concave, the acoustic impulse that each produces must itself be focused on the target concretion. Flat piezoelectric elements cannot themselves be individually focused. By mounting spherically concave piezoelectric elements in an annular array around at least a portion of the circumference of the reflector used to focus the primary shock wave source impulse, it has been found possible to control the collapse of the cavitation bubbles that were produced by the primary shock wave source when the annular array of piezoelectric generators is oriented on the circumference of the primary shock wave source reflector. This orientation combined with the spherically concave nature of the piezoelectric element produces a strong acoustic impulse at the common convergence spot of these spherically concave piezoelectric elements. This common convergence spot should be essentially congruent with the focus of the primary shock wave source.

To achieve control and collapse of the cavitation bubbles produced by the primary shock wave source, it is necessary to operatively connect the piezoelectric generators to a time delay generator so that the release of the auxiliary shock waves is delayed and occurs after the maximum pressure of the primary shock wave has been produced at its focus. At least one hydrophone aligned essentially confocally with the primary shock wave source determines the needed time delay. By these means, it has been found possible to control and to force collapse of the cavitation bubbles produced by the primary shock wave source so that they are forced to collapse towards the targeted renal concretions in vivo and to produce simultaneously improved concretion comminution and reduced tissue injury. For this purpose, the plurality of piezoelectric generators should comprise between 2 and 2000 piezoelectric elements although six piezoelectric elements may be advantageous in terms of the combined consideration of economics and physical effects. These combined piezoelectric generators should provide a peak pressure between 9 and 30 MPa near the target concretions in order to be effective, and in addition, should produce this peak pressure with a time delay within the range of 10 to 1000 μs after the peak pressure of the primary shock wave source is produced, although 401 to 1000 μs can be advantageous in certain cases. Finally, the primary shock wave source should produce a peak pressure between 20 and 130 MPa and have a tensile component with a pulse duration between 2 and 10 μs and a compressive component with a pulse duration between 0.5 and 3 μs in order to generate a profusion of cavitation bubbles.

It will be understood that various details of the invention may be changed without departing from the scope of the invention. Furthermore, the foregoing description is for the purpose of illustration only, and not for the purpose of limitation, as the invention is defined by the claims as set forth hereinafter.

1. An improved electrohydraulic shock wave lithotripter apparatus for comminuting renal concretions, said improved electrohydraulic shock wave lithotripter apparatus comprising:
(a) a primary shock wave source, said primary shock wave source having a reflector operatively associated therewith, said primary shock wave source having a focus, said focus essentially coinciding with said renal concretions, said primary shock wave source having a maximum pressure, said primary shock wave source producing cavitation bubbles around said focus of said primary shock wave source, said reflector having a circumference;
(b) a plurality of piezoelectric generators for producing auxiliary shock waves, said plurality of piezoelectric generators each having a common convergence spot, each piezoelectric generator consisting essentially of at least one substantially spherically concave piezoelectric element, said piezoelectric generators being operatively associated with at least a portion of said circumference of said reflector, said annular array of said plurality of said piezoelectric generators being oriented on said circumference of said reflector to produce convergence of each said spherically concave piezoelectric element at said common convergence spot, said common convergence spot being essentially congruent with said focus of said primary shock wave source;
(c) said primary shock wave source being operatively connected to a time delay generator, said time delay generator delaying said auxiliary shock waves by a time delay, said auxiliary shock waves having a peak pressure, said peak pressure of said auxiliary shock waves being delayed by said delay generator so that said peak pressure of said auxiliary shock waves occurs between 10 and 1000 μs after said maximum pressure of said primary shock wave source to control and to force collapse of said cavitation bubbles produced by said primary shock wave source; and
(d) at least one hydrophone aligned essentially confocally with said primary shock wave source to determine said time delay, wherein said cavitation bubbles are controlled and forced to collapse towards said renal concretions for improved concretion comminution and reduced tissue injury.

2. The apparatus according to claim 1 wherein said plurality of piezoelectric generators comprises 2 and 2000 piezoelectric elements.

3. The apparatus according to claim 2 wherein said plurality of piezoelectric generators comprises six piezoelectric elements.

4. The apparatus according to claim 1 wherein said plurality of piezoelectric generators provides a peak pressure of about 9 and 30 MPa.

5. The apparatus according to claim 4 wherein said plurality of piezoelectric generators produces said peak pressure between 401 and 1000 μs after peak pressure of the primary shock wave source is produced.

6. The apparatus according to claim 1 wherein said primary shock wave source a peak pressure between 20 and 130 MPa.

7. The apparatus according to claim 1 wherein said primary shock wave source comprises a tensile component of the primary shock wave between 2 and 10 μs and a compressive component of the primary shock wave of 0.5 and 3 μs.

8. The apparatus of claim 1 additionally comprising at least one self-focused hydrophones aligned confocally with said primary shock wave source to monitor said cavitation bubbles produced by said primary shock wave source.

9. An improved electromagnetic shock wave lithotripter apparatus for comminuting renal concretions, said improved electromagnetic shock wave lithotripter apparatus comprising:
(a) a primary shock wave source, said primary shock wave source having an electromagnetic shock wave emitter operatively associated therewith, said primary shock wave source having a focus, said focus essentially coinciding with said renal concretions, said primary shock wave source producing cavitation bubbles around said focus of said primary shock wave source, said electromagnetic shock wave emitter having a circumference;
(b) a plurality of piezoelectric generators for producing auxiliary shock waves, said plurality of piezoelectric generators each having a common convergence spot, each piezoelectric generator consisting essentially of at least one substantially concave piezoelectric element, said piezoelectric generators being operatively associated with at least a portion of said circumference of said electromagnetic shock wave emitter, said annular array of said plurality of said piezoelectric generators being oriented on said circumference of said electromagnetic shock wave emitter to produce convergence of each said spherically concave piezoelectric element at said common convergence spot, said common convergence spot being essentially congruent with said focus of said primary shock wave source;
(c) said primary shock wave source being operatively connected to a time delay generator, said time delay generator delaying said auxiliary shock waves by a time delay, said auxiliary shock waves having a peak pressure, said peak pressure of said auxiliary shock waves being delayed by said delay generator so that said peak pressure of said auxiliary shock waves occurs between 10 and 1000 μs after said maximum pressure of said primary shock wave source to control and to force collapse of said cavitation bubbles produced by said primary shock wave source; and
(d) at least one hydrophone aligned essentially confocally with said primary shock wave source to determine said time delay, wherein said cavitation bubbles are controlled and forced to collapse towards said renal concretions for improved concretion comminution and reduced tissue injury.

10. The apparatus according to claim 9 wherein said plurality of piezoelectric generators comprises between 2 and 2000 piezoelectric elements.

11. The apparatus according to claim 10 wherein said plurality of piezoelectric generators comprises six piezoelectric elements.

12. The apparatus according to claim 9 wherein said plurality of piezoelectric generators provides a peak pressure of between 9 and 30 MPa.

13. The apparatus according to claim 12 wherein said plurality of piezoelectric generators produces said peak pressure between 401 and 1000 μs after peak pressure of the primary shock wave source is produced.

14. The apparatus according to claim 9 wherein said primary shock wave source produces a peak pressure between 20 and 130 MPa.
